(19) United States
(12) Patent Application Publication
(10) Pub. No.: US 2012/0245190 A1
(43) Pub. Date: Sep. 27, 2012

(54) AUTOPHAGY INDUCING COMPOUND AND THE USES THEREOF

(75) Inventors: Min LI, Hong Kong (HK); Jia Hong LU, Hong Kong (HK); Siva Sundara Kumar DURAIRAJAN, Hong Kong (HK); Liang Feng LIU, Hong Kong (HK)

(73) Assignee: HONG KONG BAPTIST UNIVERSITY, Hong Kong (HK)

(21) Appl. No.: 13/420,628

(22) Filed: Mar. 15, 2012

Related U.S. Application Data
(60) Provisional application No. 61/466,479, filed on Mar. 23, 2011.

(51) Int. Cl. A61K 31/437; A61P 25/28; A61P 25/16; A61P 35/00; A61P 21/00; A61P 31/00; A61P 25/20; A61P 43/00; C07D 471/20; A61P 25/00

(52) U.S. Cl. 514/278; 546/15

(57) ABSTRACT

The present invention relates to a composition comprising compounds of formula (I-V),

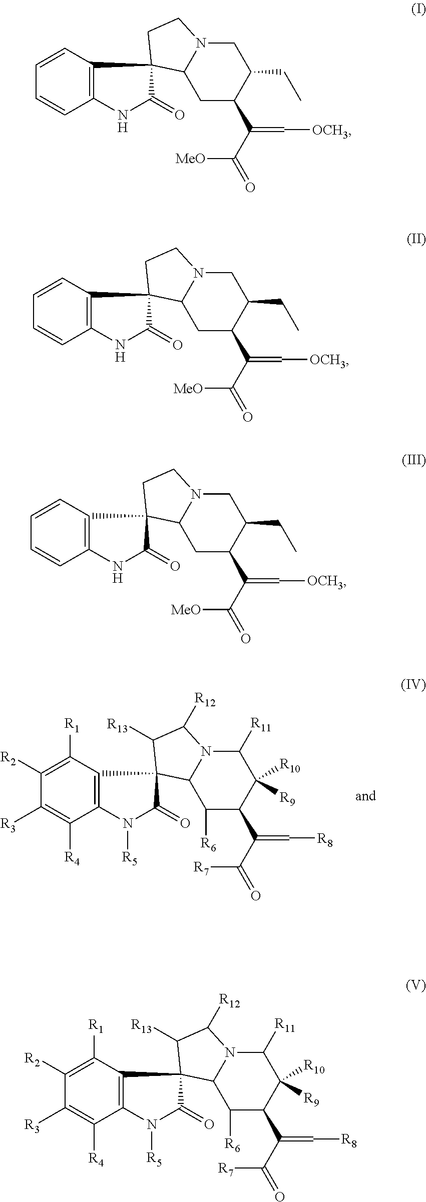

the pharmaceutically-acceptable carrier, solvent, or the salts thereof which is used to treat autophagy-associated diseases such as Alzheimer's disease, Parkinson's disease, Huntington's disease, etc. The present invention also relates to a method of treating these diseases by administering a therapeutically-effective amount of the compound to the subject in need of the treatment. The present invention further relates to the use of this compound in preparation of the composition to treat the diseases.

### CROSS-REFERENCE TO RELATED APPLICATIONS

The present application claims priority of U.S. provisional application No. 61/466,479 filed Mar. 23, 2011, and which the disclosure is hereby incorporated by reference.

### TECHNICAL FIELD

The present invention relates to a composition including an autophagy inducing compound. In particular, the present invention relates to a composition including the autophagy inducing compound used to degrade abnormal protein deposit in the nervous system by inducing autophagy and related methods of treatment, such as treating neurodegenerative diseases associated with abnormal protein aggregation and/or deposition and cancer.

### BACKGROUND

Macroautophagy, herein referred to as autophagy, is a highly conserved process for cellular degradation and recycling of cytosolic contents to maintain cellular homeostasis. Autophagy substrates are generally cellular organelles, long-lived proteins and aggregate-prone proteins. Due to its functionality to clear cytosolic contents, this highly conserved process has been shown to be a promising approach for treatment of diseases characterized by the formation of intracellular aggregates, such as aging of the brain and neurodegeneration. Dysfunction of the autophagy pathway has also been implicated in various cancers.

Aggregate-prone disorders are characterized by the formation of intracellular aggregates in specific tissues. For example: neurodegenerative diseases are associated with the accumulation of abnormal protein aggregates in affected regions of the brain. One example of a disease-causing, aggregate-prone protein is alpha-synuclein (α-syn). Over-expression of α-syn due to duplication or triplication of the α-syn gene locus has been shown to result in familial form of Parkinson's disease (PD). Point mutations (A53T and A30P) of α-syn increase the aggregation propensity thereof also lead to early onset of familial PD. Moreover, over-expressions of wild type (WT) and mutant α-syn in transgenic mice as well as transgenic flies have been found to cause progressive locomotor defects with dopaminergic neuron loss and intracytoplasmic inclusions. It is also believed that accumulation of α-syn oligomers, which are intermediates of fibrillar aggregates or inclusion formation are toxic and lead to direct neuronal death. These findings illustrate that α-syn as valuable therapeutic target for the treatment of PD and other synucleinopathies.

Other examples of aggregate-prone disorders include Alzheimer's disease; Hungtinton's disease; spinocerebellar ataxia types 1, 2, 3, 6, 7 and 17; spinobullar muscular atrophy; dentatorubral-palli-doluysian atrophy; different forms of dementia that are caused by mutations in the neuronal protein tau; forms of motor neuron disease caused by mutations in superoxide dismutase 1 (SOD1) and forms of peripheral neuropathy caused by mutations in peripheral myelin protein 22 (PMP22).

Apart from α-syn, it is well-established that other large disease-causing protein aggregates like oliogomeric α-syn, tau and mutant huntingtin, are also relied greatly on autophagy pathway for clearance since they cannot go through the narrow core of proteasomes for degradation. Furthermore, recent reports using mutant mice lacking the autophagy-related genes atg5 or atg7 indicate basal autophagy has an important role in neuronal functions.

Certain bacterial and viral infections may also be treatable by autophagy upregulation, since the pathogens can be engulfed by autophagosomes and transferred to lysosomes for degradation. For instance: Mycobacterium tuberculosis; Group A Streptococcus and Herpes Simplex Virus Type I.

Approaches to activate autophagy for therapeutic applications, such as treating neurodegenerative diseases and cancers have been explored in the art. For instance: Bradner et al. (WO2008/122038) discloses various modulators of autophagy such as compounds with a bis-indolyl maleimide core for the treatment or prevention of neurodegenerative diseases, proliferative diseases as well as infectious diseases; Rubinsztein et al. (US20070155771) describes the use of rapamycin for the treatment of conditions characterized by formation of intracellular protein aggregates by stimulation of autophagic activity and Yuan et al. (US2010/0267704) discloses treatments using autophagy inducing compounds including Loperamide, Amiodarone, Niguldipine, Pimozide.

However, current small molecules which upregulate autophagy in mammalian brains such as rapamycin, are specific mTOR inhibitors. TOR proteins are known to control several cellular processes besides autophagy in organisms from yeast to human. Thus, long-term use of these mTOR dependent small molecule autophagy inducers is likely to contribute to complications. Moreover, autophagy in the central nervous system is also known to be regulated differently from that in non-neuronal cells and the induction thereof in neuronal cells has been shown to be more difficult than in non-neuronal cells. These classical autophagy inducers either fail to induce autophagy in the cortex of mouse brains or induce only mild autophagy in neurons.

Isorhynchophylline (IsoRhy), one of the Uncaria oxindole alkaloids has been used as a component of various compositions to induce various biological outcomes, such as protective effects on ischemia-induced neuronal damage; inhibition of Listeriolysin O-induced nitric oxide and endothelin-1 release and prevention angiotensin II induced proliferation. Nevertheless, there is no teaching or suggestion in the prior art relating to this kind of compounds (oxindole alkaloids) to induce autophagy.

Accordingly, there is a need for a potent agent which specifically induces autophagy independent of mTOR in neurons for the treatment of diseases that can benefit from autophagy, including but not limited to neurodegenerative disorders, immunological diseases, cardiac diseases and cancer.

### SUMMARY OF THE INVENTION

The present invention relates to novel application of a compound of formula (I),

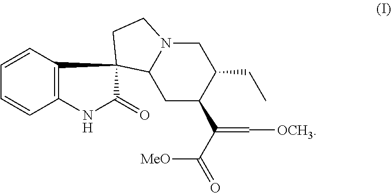

Applicants of the subject application are the first to demonstrate that the compound of formula (I) is a potent autophagy inducer and capable of degrading abnormal cytosolic contents, especially aggregate-prone proteins in neurons, thereby treating diseases that can benefit from autophagy, such as neurodegenerative diseases and cancer.

The first aspect of the present invention relates to a pharmaceutical composition comprising the compounds of formula (I) (also called Isorhynchophylline (IsoRhy)) and a pharmaceutically acceptable salt thereof, that is used for treatment of diseases that can benefit from degradation of cytoplasmic proteins, organelles or pathogens by inducement of autophagy. In particular, the compound of formula (I) induces autophagy in neurons. The compound of formula (I) of the present invention is a kind of tetracyclic oxindole alkaloid isolated from Uncaria species including, but not limited to Uncaria rhynchophylla, Uncaria macrophylla Wall, Uncaria sinensis (Oliv.) Havil and Uncaria tomentosa. The functional groups of this compound may be substituted by a moiety including but not limited to hydrogen, —CH₃, and glucose known to a skilled artisan, wherein the autophagy induction activity is maintained. The compound itself may be modified such that commonly used carriers, salts or esters known to one skilled in the art (e.g. methyl acetate, ethyl acetate) can be incorporated therein to allow different modes of administration. This compound is also small enough to pass through the blood-brain barrier in order to target specific cells/tissue in the nervous system, where abnormal protein aggregation and/or deposition occur. Evidences of the ability of such compound to pass through the blood-brain barrier is also presented in “The distribution of isorhynchophyll ine in the tissues of the rats and the determination of its plasma half-life time”, ACTA ACADEMIAE MEDICINAE ZUN YI. 2001, 24:119-120.

The present invention also relates to other tetracyclic oxindole alkaloids isolated from Uncaria species including, but not limited to corynoxine (formula II) and corynoxine B (formula III) as autophagy inducers for treating a disease that can benefit from autophagy:

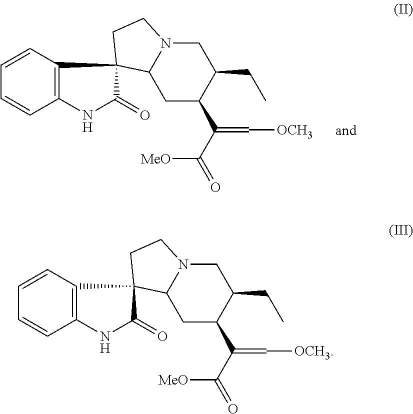

In another aspect, the present invention features a pharmaceutical compositions comprising an autophagy inducing compound in an amount effective for treating a disease that can benefit from autophagy, wherein the compound is at least one compound selected from the group including:

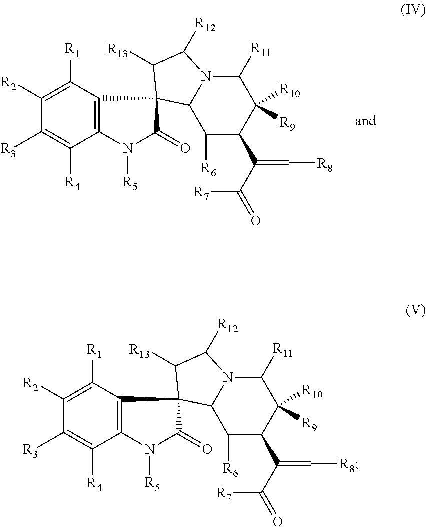

A compound of formula IV or formula V, wherein R₁, R₂, R₃, R₄, R₅, R₆, R₁₁, R₁₂ and R₁₃ are each independently selected from hydrogen, hydroxyl, halogen, C1-6 alkyl and C1-6 haloalkyl; R₇ and R₈ are each independently selected from methoxyl and hydroxyl; R₉ and R₁₀ are each independently selected from hydrogen, hydroxyl, halogen, C1-6 alkyl.

The present invention includes one or more other therapeutic agent(s) known to treat a disease that can benefit from inducement of autophagy, such as chemotherapeutic agents known in the art; or a compound that may potentiate the autophagy inducing activity of a compound of formula (I-V). The present invention further comprises one or more of a pharmaceutically-acceptable carrier, solvent, excipient, adjuvant and/or prodrug.

The second aspect of the present invention relates to methods for treatment of diseases that can benefit from inducement of autophagy by administration of a therapeutically effective amount of the pharmaceutical composition of the present invention to a subject in need thereof. In one embodiment of this aspect, the disease is caused by abnormal protein aggregation and/or deposition in the nervous system, especially among the neuronal cells. In another embodiment, the disease is cancer, wherein the induction of autophagy would inhibit cell growth or remove organelles damages by reactive oxygen species, such as mitochondria or tumor cells and the autophagy target is cancerous cells or tumor cells. In another embodiment of this aspect, the method further comprises administering one or more other therapeutic agent known to treat diseases that benefit from inducement of autophagy.

The third aspect of the present invention relates to a method of using the compounds of formula (I-V) in the preparation of a pharmaceutical composition for treating diseases that can benefit from autophagy enhancement.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Chemical structure of Isorhynchophylline (IsoRhy) (FIG. 1A); Western blot analysis of the expression level of autophagy marker, LC3-II, in different neuronal cell lines including N2a (FIG. 1B), PC12 (FIG. 1C) and SH-SY5Y (FIG. 1D) induced by 0-25 μM IsoRhy for 24 hours; Fluorescent images of GFP signal (FIG. 1E) and number of GFP-LC3 puncta per cell (FIG. 1F).

FIG. 2: Western blot analysis of the expression level of autophagy marker, LC3-II, in N2a cells induced by 25 μM IsoRhy and/or 30 μM lysosome inhibitor chloroquine (CQ) for 12 hours (FIG. 2A), and the ratio of LC3-II expression to beta-actin in different treatment groups (FIG. 2B); fluorescent images of GFP signal (FIG. 2C) and number of GFP-LC3 puncta per cell (FIG. 2D) in different treatment groups including 5 mM 3-MA, 30 μM CQ, and/or 25 μM IsoRhy for 24 hours; double fluorescent images of GFP and/or RFP signals from N2a cells containing a tandem fluorescent mRFT-GFP-LC3 (Tf-LC3) construct (FIG. 2E) and number of GFP-LC3 and RFP-LC3 puncta per cell in different treatment groups (FIG. 2F) including 25 μM IsoRhy and/or 30 μM CQ for 24 hours.

FIG. 3: Western blot shows the expression level of autophagy marker, LC3-II, in primary mouse cortical neuron isolated from E17 embryonic mice and induced by 0-50 μM IsoRhy (FIG. 3A); fluorescent images of GFP signal (FIG. 3B) and number of GFP-LC3 puncta per cell (FIG. 3C) in mouse embryonic primary cortical neuronal cell induced by 50 μM IsoRhy for 24 hours.

FIG. 4: Fluorescent images of LysoTracker red staining of L3 Drosophila larvae fat body in different treatment groups for 6 hours (FIG. 4A); number of LysoTracker red-positive spots per field in different treatment groups (FIG. 4B).

FIG. 5: Western blot analysis of expression level of WT α-syn (FIG. 5A), mutants α-syn A30T (FIG. 5B) and A53P (FIG. 5C), GFP control (FIG. 5D) WT α-syn in N2a cells with treatment of 25 μM IsoRhy or 5 mM 3-MA and 30 μM CQ (FIG. 5E) and the expression level of WT α-syn as compared to control in different treatment groups (FIG. 5F); schematic diagram of a bimolecular fluorescence complementation-based cell model for visualizing the degradation of α-syn oligomer by IsoRhy (FIG. 5G), and comparison in GFP signal in different treatment groups (FIGS. 5H and 5I); western blot analysis of high molecular weight α-syn oligomer species (FIG. 5J); fluorescent images of co-expressed α-syn oligomer- and synphilin-1-GFP signal in N2a cells (FIG. 5K) and percentage of cells with GFP signal which is proportional to the percentage of aggresome formation (FIG. 5L) in different treatment groups.

FIG. 6: Double fluorescent images of HA-staining for α-syn expression and tyrosine hydroxylase (TH) staining of human DA neurons differentiated from embryonic stem cells (FIG. 6A); western blot analysis of both WT and A53T α-syn expression levels in differentiated DA neurons (FIG. 6B).

FIG. 7: Western blot analysis of expression level of phosphorylated mTOR (p-mTOR) or its substrate P70S6K (p-P70S6K) in N2a cells with treatment of 25 μM IsoRhy or 0.2 μM rapamycin for 6 hours (FIG. 7A); western blot analysis of Beclin 1 expression in N2a cells with non-target or Beclin 1-specific siRNA treatments followed by IsoRhy (FIG. 7B)

FIG. 8: Western blot analysis of the LC3II expression level in N2a cells treated with different oxindole alkaloids; Isorhynchophylline (IsoRhy) (FIG. 8A), Corynoxine (Cory) (FIG. 8B) and Corynoxine B (Cory B) (FIG. 8C) for 12 hours.

### ABBREVIATIONS

α-syn: alpha-synuclein;

BiFC: Bimolecular Fluorescence Complementation

CQ: chloroquine;

DA: dopaminergic;

GFP: enhanced green fluorescent protein;

HA: hyaluronan

IsoRhy: Isorhynchophylline;

3-MA: 3-Methylamphetamine;

(MAP)LC3: microtubule-associated protein 1 light chain 3;

mTOR: mammalian target of rapamycin;

PD: Parkinson's disease;

Tf-LC3: tandem fluorescent LC3

RFU: Relative Fluorescence Unit

RFP: Red Fluorescence Protein

### DEFINITIONS

“a,” “an,” and “the” as used herein include “at least one” and “one or more” unless stated otherwise. Thus, for example, reference to “a pharmacologically acceptable carrier” includes mixtures of two or more carriers as well as a single carrier, and the like.

The terms “aggregate-prone proteins” and “autophagy substrate” are used interchangeably, referring to cytosolic proteins that are prone to aggregation and deposition and their aggregation are disease causing. Examples include, but are not limited to α-synuclein, Huntingtin, tau, SOD1 and PMP22 and the mutant and variant forms thereof.

The term “autophagy” refers to macroautophagy, unless stated otherwise, which is the catabolic process involving the degradation of a cell's own components; such as, long lived proteins, protein aggregates, cellular organelles, cell membranes, organelle membranes, and other cellular components. The mechanism of autophagy may include: (i) the formation of a membrane around a targeted region of the cell, separating the contents from the rest of the cytoplasm, (ii) the fusion of the resultant vesicle with a lysosome and the subsequent degradation of the vesicle contents. The term autophagy may also refer to one of the mechanisms by which a starving cell re-allocates nutrients from unnecessary processes to more essential processes. Also, for example, autophagy may inhibit the progression of some diseases and play a protective role against infection by intracellular pathogens.

The diseases that benefit from autophagy inducement are those that can be treated by the inventions as disclosed herein. The diseases include aggregate-prone disorder which represents any disease, disorder or condition associated with or caused by abnormal protein aggregates that are not sufficiently destroyed by a natural autophagy process in an organism and can be treated through degradation thereof via induction of autophagy by the subject invention. For example, such diseases include Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease. The diseases also include cancer e.g., any cancer wherein the induction of autophagy would inhibit cell growth and division, reduce mutagenesis, remove mitochondria and other organelles damaged by reactive oxygen species or kill developing tumor cells. They can be chronic diseases which refers to persistent and lasting diseases, medical conditions or diseases that have developed slowly. The diseases that can be treated by the subject invention also include, but not limited to, cardiovascular disorders, autoimmune disorders, metabolic disorders, hamartoma syndrome, genetic muscle disorders, and myopathies.

The term “autophagy inducing compound” refers to a compound that induces autophagy in a cell. The term autophagy inducing compound, as used herein, comprises the compound disclosed herein as well as the variants, isomers, metabolites or derivatives thereof.

The term “pharmaceutically acceptable carrier” refers to any carriers known to those skilled in the art to be suitable for a particular mode of administration. For example, carriers may include one or more solvents, dispersion media, diluents, adjuvants, excipients, vehicles, coatings, antibacterial and antifungal agents, isotonic and absorption delaying agents and the like, that are compatible with the compounds of the present invention. In addition, the compounds of formula (I-V) or salt and derivative thereof can be mixed with other active materials that do not impair the desired action, or with materials that supplement the desired action, or have another action. The autophagy inducing compounds may be formulated as the sole pharmaceutically active ingredient in the composition or may be combined with other active ingredients.

The phrase “therapeutically effective amount” refers to an amount of the compound of the present invention being sufficient to show benefit or clinical significance to an individual. Those skilled in the art would appreciate the actual amount or dose administered, and time-course of administration, will depend on the nature and severity of the diseases being treated, the age and general condition of the subject being treated as well as the mode of administration and so forth.

### DETAILED DESCRIPTION OF THE INVENTION

The present invention pertains to novel therapeutic applications of compounds of formula (I-V):

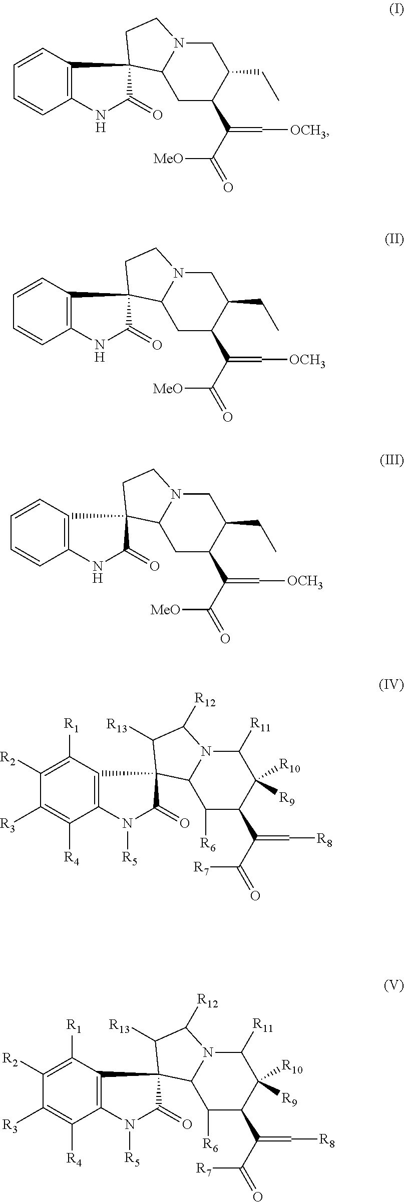

which function as autophagy inducers. In particular, the compounds of formula (I-V) are potent autophagy inducers in neuronal cells.

The present invention also provides methods and compositions useful for inducing autophagy that includes a therapeutically effective amount of the compounds of formula (I-V), and a pharmaceutically acceptable salt thereof. The compound of formula (I) is also an mTOR-independent and beclin 1-dependent autophagy inducer that is capable of promoting maturation of autophagosome in autophagy for degrading abnormal proteins that are prone to aggregation. In one embodiment, the compound of the present invention and the composition containing the compound of the present invention are able to pass through blood brain barrier to induce autophagy, thereby degrading protein aggregates in cells/tissues of the nervous system. In this embodiment, the cells/tissues in the nervous system are selected from the group consisting of but are not limited to cortical neurons, hippocampus neurons, Thyrosine hydrolase positive neurons, glial cells. In one embodiment, the abnormal proteins that can be degraded by autophagy induced by the compound and composition of the present invention include but are not limited to α-syn, huntingtin, tau, SOD1, PMP22, ataxin, synphilin 1, and variants and mutated forms thereof and any other disease causing aggregate-prone proteins. In a preferred embodiment, the present invention is able to degrade wild-type and mutant forms of α-syn monomers, wild-type and mutant forms of α-syn oligomers and wild-type and mutant forms of α-syn and synphilin-1 aggresomes.

In one embodiment, the compounds of formula (I-V) are tetracyclic oxindole alkaloids, isolated from Uncariae species; or can be synthesized by chemistry method. In another embodiment, the compound of formula (I) is Isorhynchophylline (IsoRhy), the compound of formula (II) is corynoxine; the compound of formula (III) is corynoxine B.

In one embodiment, the diseases that can be treated by the compounds and composition of the present invention are those that can benefit from autophagy inducement. For example: aggregate-prone diseases that are caused by abnormal aggregation and/or deposition of aggregate-prone proteins, wherein the autophagy promotes the clearance of protein aggregation. These aggregate-prone diseases include but not limited to Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease.

In another embodiment, diseases also include cancer, wherein the induction of autophagy would inhibit abnormal cell growth and division, reduce mutagenesis, and remove mitochondria and other organelles damaged by reactive oxygen species or kill developing tumor cells. The cancer may include but not limited to cancer of the breast, liver, prostate, stomach, colon, GI tract, pancreases, skin, head, neck, throat, bladder, eye, esophagus, lung, kidney, or brain.

In yet another embodiment, diseases that benefit from autophagy can be chronic diseases which refer to a persistent and lasting disease, medical condition or one that has developed slowly. In yet another embodiment, the diseases also include cardiovascular disorders, autoimmune disorders, metabolic disorders, hamartoma syndrome, genetic muscle disorders, and myopathies. Examples of diseases that benefit from autophagy are disclosed in WO2010/129681 and US2010/0267704, the disclosures of which are incorporated herein by reference in their entirety. Moreover, infections wherein pathogens or pathogen proteins are degraded by autophagosomes and transferred to lysosomes for degradation are susceptible to treatment with autophagy inducer. For example tuberculosis, Group A Streptococcus infections, and viral infections (e.g., herpes simples virus type I) may be treated according to the present invention.

The compounds and the compositions of present invention may be administered alone or in combination with one or more other therapeutic agent(s) known to treat diseases that can benefit from autophagy, such as rapamycin; or a compound that may potentiate the autophagy inducing activity of the compounds of formula (I-V). In some embodiments, where the treatment of disease is cancer, the present invention may be administered in conjunction with chemotherapeutic agents that are known in the art. Examples of chemotherapeutic agents that may be used in conjunction with the present invention are described in US2011/0014303, the disclosure of which is incorporated herein by reference in its entirety. Further, the compounds of the present invention can be affiliated with monoclonal antibodies to various cancer antigens or aggregate-prone proteins such that the autophagy-inducing properties are directed to cancer cells or cells where abnormal protein aggregation and/or deposition occur.

In another embodiment, the composition of the present invention additionally includes a pharmaceutically acceptable carrier, excipient, buffer, stabilizer or other materials known to those skilled in the art to be suitable for administration to living organisms. Such materials should be neither toxic, interfere with nor impair the efficacy of the compounds of the present invention. The materials may have another effect or supplement the autophagy inducing activity of the compounds of present invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; Cremophor; Solutol; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of those skilled in the art.

The method of treatment for the diseases that benefit from autophagy of the present invention includes administering a therapeutically effective amount of the compounds of the present invention or the composition containing the compound of the present invention to a subject in need thereof, where the subject is an animal including a human. Methods of the present invention further include administering the one or more therapeutic agent(s) in conjunction with the compounds or the composition of the present invention. The mode of administration of the composition of the present invention includes topical, parenteral, intravenous, intra-arterial, subcutaneous, intramuscular, intracranial, intraorbital, ophthalmical, intraventricular, intracapsular, intraspinal, intracisternal, intraperitoneal, intranasal, by aerosol, by suppositories, or by oral delivery. The compositions may be administered independently or in combination with other compositions if necessary. The compositions may also be prepared in different forms such as cream, gel, lotion, solution, solid, tablet, capsule, powder, paste, aerosol, etc depending on the desired modes of administration.

Additionally, it will be apparent to those of ordinary skill in the art that the course of treatment, such as, the number of doses of the composition given per day for a defined number of days will principally be determined by the nature and extent of the condition being treated, the form, route and site of administration, and the individual being treated. Suitable conditions can be determined by conventional techniques.

The present invention is further illustrated by the following working examples, which should not be construed as further limiting. While the working examples merely rely upon on the clearance of alpha-synuclein and its variants by IsoRhy, the working examples are intended to demonstrate the autophagy induction ability to degrade aggregate-prone proteins, it is to be understood that other aggregate-prone proteins can also be cleared by the claimed invention.

### EXAMPLES

In the following examples, the following materials are used; various commercial sources for the materials are provided. Details of the various protocols are also set forth below:

Reagents and Antibodies.

Isorhynchophylline (APC-164) was purchased from Aktin Chemicals. 3-MA (M9281) and chloroquine (C6628) were purchased from Sigma-Aldrich. Rapamycin (R5000) was purchased from LC Laboratories. LysoTracker Red DND-99 (L-7528), goat anti-mouse (626520) and goat anti-rabbit (G21234) secondary antibodies were purchased from Invitrogen. Anti-β-actin (sc-47778), anti-GFP (sc-8334) and anti-tyrosine hydroxylase (sc-14007) antibodies were purchased from Santa Cruz Biotechnology. Anti-LC3 (2775), anti-phospho-mTOR (5536), anti-phospho-p70S6K (9234), anti-(3738) antibodies were purchased from Cell Signaling Technology. Anti-α-syn antibody (610786) was purchased from BD Transduction Laboratories. The instant Drosophila food (173212) was purchased from Carolina Biological Supply Company.

Cell Lines and Cell Culture.

N2a and SH-SY5Y cells were maintained in DMEM, supplemented with 10% FBS. PC12 cells were grown in DMEM (12800017, Invitrogen), supplemented with 10% FBS (10099141, Invitrogen) and 5% horse serum (16050122, Invitrogen). N2a cells constitutively expressing GFP-LC3 were selected using 800 μg/ml G418 (10131027, Invitrogen) and maintained in 200 μg/ml G418.

Primary Neuron Culture.

The E17 embryonic pup brains were dissected and cortices were placed in a Petri dish containing ice cold EBSS, with meninges carefully removed. Tissues were digested in 5 ml of digestion solution (EBSS containing 0.5 mM EDTA (E6758, Sigma), 0.5 mg/ml Papain (3120, Worthington Biochem) and 4 mg/ml L-Cysteine (C7880, Sigma)) at 37° C. for 15 minutes. The tissue was sucked out in 1 ml of digestion solution and mixed with 3 ml of digestion inhibition solution (EBSS containing 5 mg/ml BSA (A2153, Sigma), 5 mg/ml Trypsin inhibitor (T9253, Sigma) and 10 μg/ml DNase (DN25, Sigma)). The mixed tissue solution was thoroughly mixed to dissociate the cells and then transferred to a 50 ml tube through a 70 μm filter. The cells were collected by centrifugation and re-suspended in seeding medium (DMEM containing 10% FBS and 10% horse serum). The cells were seeded on poly-D-lysine treated plates, at low density for imaging (1×10⁵ cells/well of 12-well plate), or at a high density for biochemistry analysis (3×10⁶ cells/well of 6-well plate). Four hours later, seeding medium was removed and replaced with neuorbasal medium (21103049, Invitrogen) supplemented with B-27 supplement (0080085SA, Invitrogen). Two days after culture, 5 μM Ara-C(C6645, Sigma) was added. Half of the medium was changed 24 hours later. Cultures were fed every 3 days by replacing half of the old media with fresh media. Cultures were maintained for at least one week for neuron maturation.

Differentiation of Stem Cells into Dopaminergic Neurons.

The human embryonic stem (ES) cells were differentiated into dopaminergic neurons according to a previously described protocol with minor changes. Initially, ES cells were digested with dispase (17105041, Invitrogen,) and broken into smaller clusters to form the embryonic bodies. The next day, undifferentiated floating ES cell aggregates were transferred to a new flask. The cells were maintained in DMEM/F12 medium (11320082, Invitrogen) with half medium changed every day for 3 days. On the fourth day, ES cell aggregates were collected by centrifuge and re-suspended in NSM (DMEM/F12 containing 1% N2 supplement (17502048, Invitrogen), 1 μg/ml Heparin (H3149, Sigma), 200 μM NEAA (11140050, Invitrogen) and 2 mM L-glutamine (25030081, Invitrogen)) supplemented with 10% FBS. Embryonic stem cells were transferred to a new flask and medium was changed every other day. Three days later, cell aggregates were transferred to 6-well-plates. The next day, the FBS containing media were replaced with NSM containing 20 ng/ml FGF8 (PHG0184, Invitrogen) and 100 ng/ml SHH (PMC2095, Invitrogen), media were changed every other day. Five days later, the colonies in the dish were detached by pipetting gently with a P1000 pipette. Cells were collected by centrifugation and resuspended in NSM containing 50 ng/ml FGF8, 100 ng/ml SHH, 2% B27, 200 μM NEAA and transferred to a new flask. Media were changed every other day. Six days later, neurospheres were collected and digested in 200 μl accutase (A1110501, Invitrogen)/trypsin (25300062, Invitrogen) (1:1) for 3 minutes. Digestion was stopped by adding 200 μl trypsin inhibitor (R007100, Invitrogen) and cells were re-suspended in NDM (Neurobasal medium containing 1% N2 supplement and 2% B27 supplement (17504044, Invitrogen)) and plated onto laminin (23017015, Invitrogen) coated cover slips. The next day, 1 ml NDM supplemented with 20 ng/ml BDNF (10908010, Invitrogen), 50 ng/ml GDNF (PHC7045, Invitrogen), 50 ng/ml FGF8, 100 ng/ml SHH, 2% B27, 200 μM AA, 1 μM cAMP (A9501, Sigma), 1 μg/ml laminin and 1 ng/ml TGF133 (PHG9305, Invitrogen) was added to each well and media were changed every other day for 10 days. Successful differentiation of dopaminergic neurons was confirmed by tyrosine hydroxylase staining.

Plasmids and Transfection.

TfLC3 plasmids were a generous gift from Dr. T. Yoshimori (Osaka University, Japan). GNS and SGC plasmids were donated by Dr. Pamela J. McLean (Harvard Medical School, U.S.A.). Cells were transfected with plasmids using lipofectamine 2000 (11668019, Invitrogen) according to the manufacturer's protocol.

Native and Denatured PAGE and Western Blotting Analysis.

Samples to be run under denaturing conditions were lysed with RIPA lysis buffer (150 mM NaCl, 50 mM Tris-HCl, 0.35% sodium deoxycholate, 1 mM EDTA, 1% NP40, 1 mM PMSF, 5 μg/ml aprotinin, 5 μg/ml leupeptin). Samples for native gels were lysed with detergent-free lysis buffer (50 mM Tris/HCl pH 7.4, 175 mM NaCl, 5 mM EDTA pH 8.0, 1 mM PMSF, 5 μg/ml aprotinin, 5 μg/ml leupeptin), and sheared 5 times through a 28-gauge needle followed by 2 times of sonication for 5 seconds according to previously described protocol. For denaturing, SDS-PAGE was performed using Tris-Glycine SDS running buffer and SDS sample buffer, and for native conditions, native-PAGE was run with detergent-free Tris-Glycine running buffer (BN2007, Invitrogen) and 4× native sample buffer (BN2003, Invitrogen) on a pre-casted native PAGE gel (BN1002BOX, Invitrogen). The proteins on the gels were then transferred to PVDF membrane (RPN3O3F, GE Healthcare) and processed for immunoblotting. Membranes were blocked with 5% non-fat milk and probed with the appropriate primary and secondary antibodies. The desired bands were visualized using the ECL kit (32106, Pierce). The band density was quantified using the ImageJ program and normalized to that of the control group.

Immunostaining.

Cells were fixed with 3.7% paraformaldehyde in PBS, immunolabeled with antibody against TH (2792, Cell Signaling Technology) or HA (2367, Cell Signaling Technology) and fluorophore-conjugated secondary antibody (Cy3-conjugated goat anti-rabbit (078-15-061, KPL) or Cy5-conjugated goat anti-mouse (072-15-18-18, KPL)), and mounted with Fluor Save reagent (345789, Calbiochem). Fluorescence was recorded using a confocal microscope.

Cell Imaging and Puncta Counting.

Cells were fixed in 3.7% paraformaldehyde (158127, Sigma) for 10 minutes and mounted with Fluor Save reagent. Cell images were recorded using a fluorescence microscope or confocal microscope. GFP or RFP Puncta number were counted in cells as described previously. Briefly, the GFP-LC3 or RFP-LC3 puncta in each cell were manually counted, and at least 50 cells were randomly selected for counting in each group. The data presented were from one representative experiment of at least 3 independent experiments.

Drosophila Culture and Drug Feeding.

Flies were raised at 25° C. on standard corn meal medium supplemented with dry yeast. IsoRhy and rapamycin were initially dissolved in DMSO then diluted in water to desired concentrations. The drug containing water was added into instant Drosophila food and mixed thoroughly. As the control, the same amount of DMSO was also mixed with instant Drosophila food. For the treatment, L3 larvae or adult flies were transferred to the drug-containing medium and incubated for indicated time.

Lysotracker Staining and Quantitative Analysis of Autophagic Structures.

L3 larvae were dissected using fine forceps under a dissecting microscope and inverted so that fat bodies were exposed to the incubating solution. The larvae carcasses were stained with 100 nM of LysoTracker red in PBS for 5 minutes at room temperature. After the incubation, the larvae carcasses were rinsed once in PBS and transferred to a glass slide with a drop of mounting medium on it. The fat bodies (one major lobe per animal) were excised, and the remaining tissue was discarded. Fat body lobes were then covered with a cover slide and immediately observed under a standard fluorescence microscope. Quantitative analysis of lysotracker-positive spots was performed according to previous described protocol with minor revision. At least 6 fat body lobes from three independent animals of each group were obtained. The numbers of lysotracker-positive spots were quantified from at least 20 randomly selected fluorescent image fields (4700 μm²/field).

### Example I

### IsoRhy Induces Autophagy in Neuronal Cell Lines

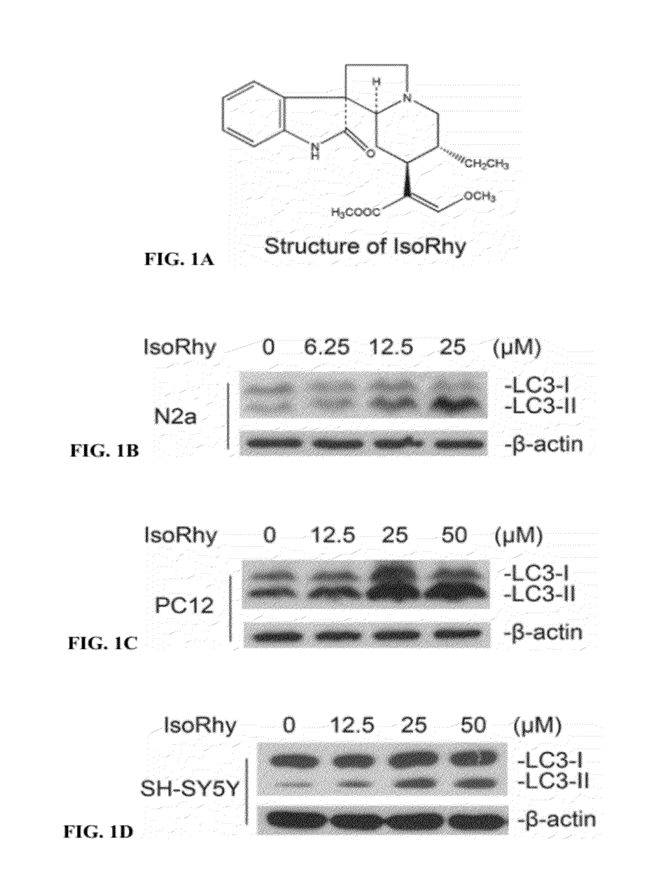

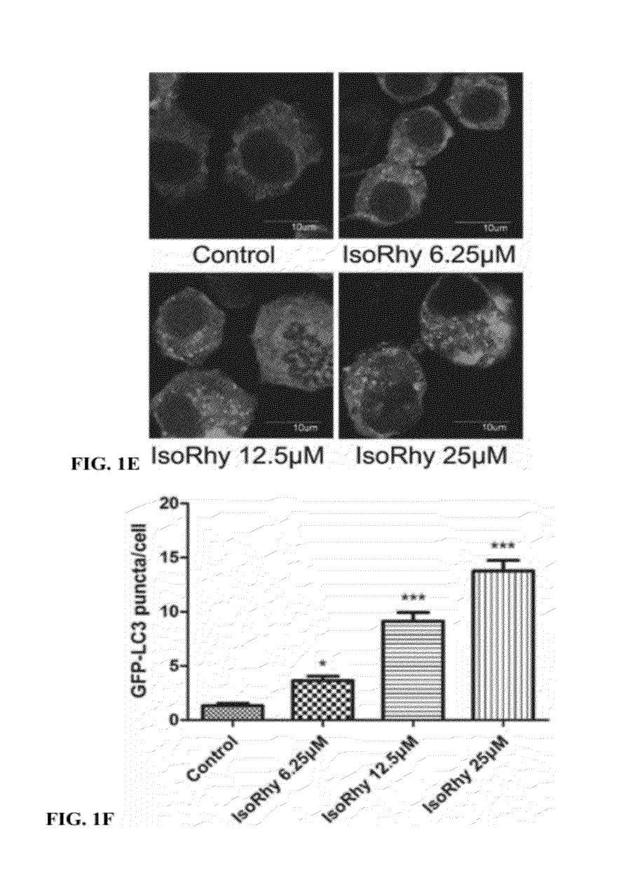

Induction of autophagy has been shown to be more difficult in neuronal cells than in non-neuronal cells. In order to confirm the neuronal autophagy inducing activity of IsoRhy (its chemical structure is shown in FIG. 1A), mouse neuroblastoma N2a, rat phenochromocytoma PC12 and human neurobastoma SH-SY5Y are treated with different concentrations of IsoRhy for 24 hours and cell lysates are subjected to western blotting analysis of LC3-II expression which is an autophagy-specific marker. It is shown that IsoRhy increases levels of LC3-II in N2a, PC12 and SH-SY5Y cells in a dose-dependent manner, without affecting LC3-I levels (FIG. 1B-D).

Further, a neuroblastoma cell line N2a constantly expressing GFP-LC3 (a standard autophagy marker protein) is established. The formation of GFP-LC3 puncta under IsoRhy treatment is observed under a confocal microscope. Data is presented as the mean±SEM of one representative experiment from three independent experiments. (*p<0.05, ***p<0.001, one-way ANOVA for multiple comparison and Tukey's test as post hoc test). The data illustrates that IsoRhy induces massive GFP-LC3 puncta formation in the N2a GFP-LC3 cells (FIG. 1E, F).

The elevated levels of LC3-II expression in various neuronal cell lines and massive GFP-LC3 puncta formation in N2a cells in the presence of IRY demonstrate that IRY is a potent autophagy inducer in neurons.

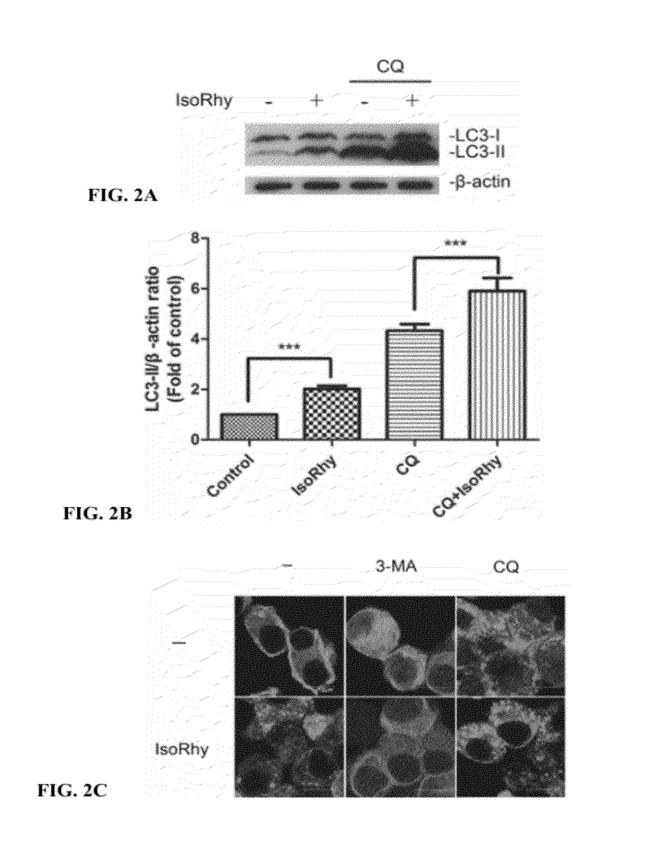

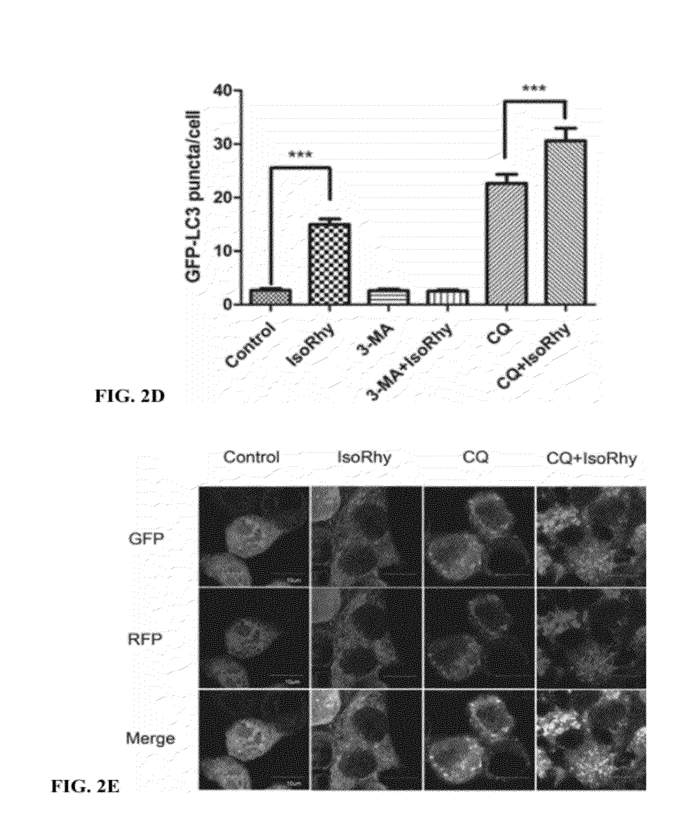

To confirm that enhancement of autophagy markers by IsoRhy is due to induction of autophagy rather than blockage of autophagosome maturation, N2a cells are treated with 25 μM IsoRhy or 30 μM lysosome inhibitor CQ together with IsoRhy for 12 hours. Cell lysates are subject to western blot analysis. Data are presented as the mean±SEM from 3 independent experiments (***p<0.001, one-way ANOVA for multiple comparison and Tukey's test as post hoc test). Both LC3-II levels and the number of GFP-LC3 puncta in the IsoRhy and CQ co-treatment group are much higher than in the CQ-alone treatment group (FIG. 2A-D). Meanwhile, IsoRhy induces GFP-LC3 puncta formation is abolished by treatment of 5 mM autophagy inhibitor 3-MA for 24 hours (FIG. 2C, D). Cells were fixed in 4% paraformaldehyde and analyzed under a confocal microscope. Data are presented as the mean±SEM of one representative experiment from three independent experiments. (***p<0.001, one-way ANOVA for multiple comparison and Tukey's test as post hoc test). The abolishment of GFP-LC3 puncta formation by 3-MA suggests that the enhancement of LC3-II and GFP-LC3 puncta formation by IsoRhy is due to its ability to induce autophagy. Thus, IsoRhy is indeed an autophagy inducer in neuronal cells.

A system established by Kimura et al. based on a tandem fluorescent mRFP-GFP-LC3 (Tf-LC3) construct is used to investigate the autophagosome maturation process. mRFP is more stable than GFP in the acidic/proteolytic condition in lysosome. Therefore, red-only puncta indicates the normal maturation of the autolysosomes. In contrast, co-localization of GFP and RFP puncta indicates impaired fusion between autophagosomes and lysosomes or disruption of lysosome function. Here, this system is utilized and establishes a pattern of GFP and mRFP fluorescence changes in N2a cells after IsoRhy treatment. Cells are fixed in 4% paraformaldehyde and analyzed under a confocal microscope. IsoRhy induces massive GFP and mRFP puncta formation after 24 hours of IsoRhy treatment. However, the number of GFP puncta is much lower as compared to mRFP puncta, indicating efficient autophagosome-lysosome maturation and degradation. In cells treated with CQ, there is similar accumulation of GFP and mRFP puncta due to lysosome inhibition. In cells treated with both CQ and IsoRhy, the number of GFP and mRFP puncta are even more than in the CQ-alone group, but GFP and mRFP puncta numbers are very similar (FIG. 2E, F). The observations further confirm that enhancement of GFP- and RFP-LC3-II puncta expression by IsoRhy are achieved via autophagy induction flux in neuronal cells rather than inhibiting lysosomal degradation of LC3-II. Results were presented as means±SEM of one representative experiment from three independent repeats (**p<0.01 compare the difference between GFP and RFP puncta in each group, two-way ANOVA for multiple comparison and Bonferroni test as post hoc test).

### Example II

### IsoRhy Induces Autophagy in Primary Mouse Cortical Neurons

To further confirm the pro-autophagic effect of IsoRhy on primary neurons, mouse primary cortical neurons isolated from E17 embryonic ICR mice are used in this study. The primary neurons are treated with different concentrations of IsoRhy for 24 hours, and autophagic marker GFP-LC3 expression is examined by Western blotting analysis. Neurons are fixed in 4% paraformaldehyde and analyzed under a confocal microscope. GFP-LC3 puncta number in each GFP positive neuron is counted and at least 20 neurons in each group is counted. Data presents as the mean±SEM of one representative experiment from three independent experiments (***p<0.001, Student t test).

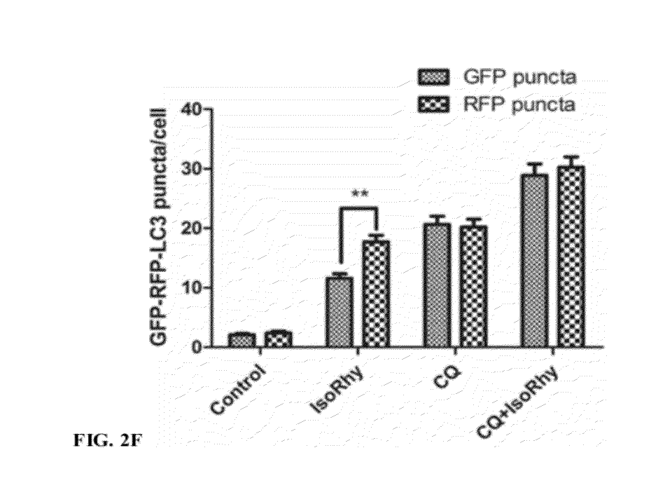

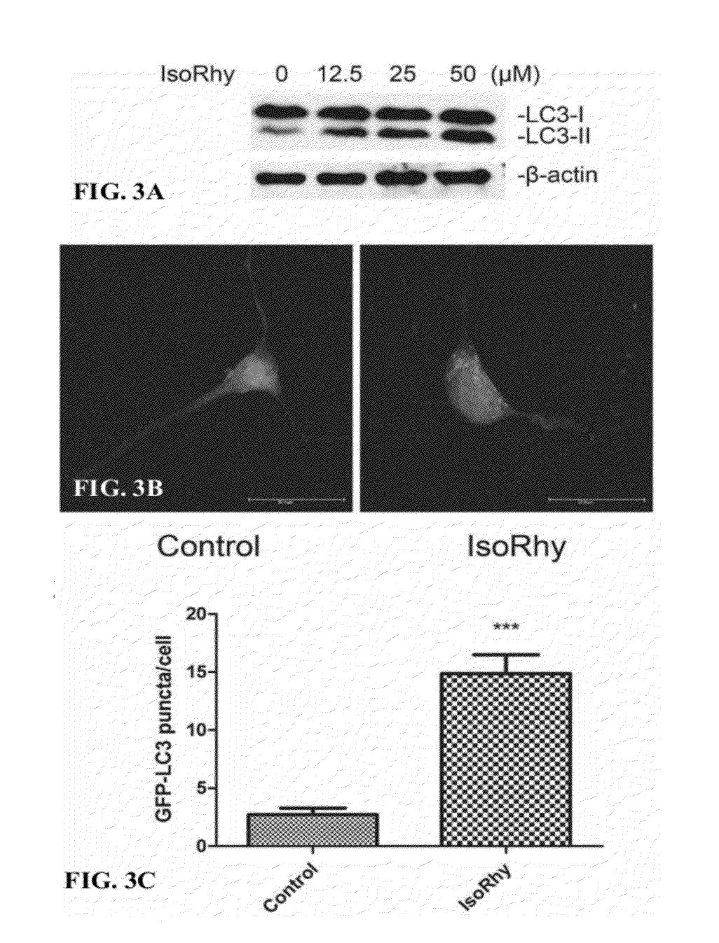

Again, a dose-dependent increase of LC3-II by IsoRhy is observed in mouse primary cortical neurons (FIG. 3A). In neurons transfected with the GFP-LC3 construct, IsoRhy induces massive formation of GFP puncta (FIGS. 3B, C). These data indicate that IsoRhy is also a potent autophagy inducer in primary neurons.

### Example III

### IsoRhy Induces Autophagy In Vivo

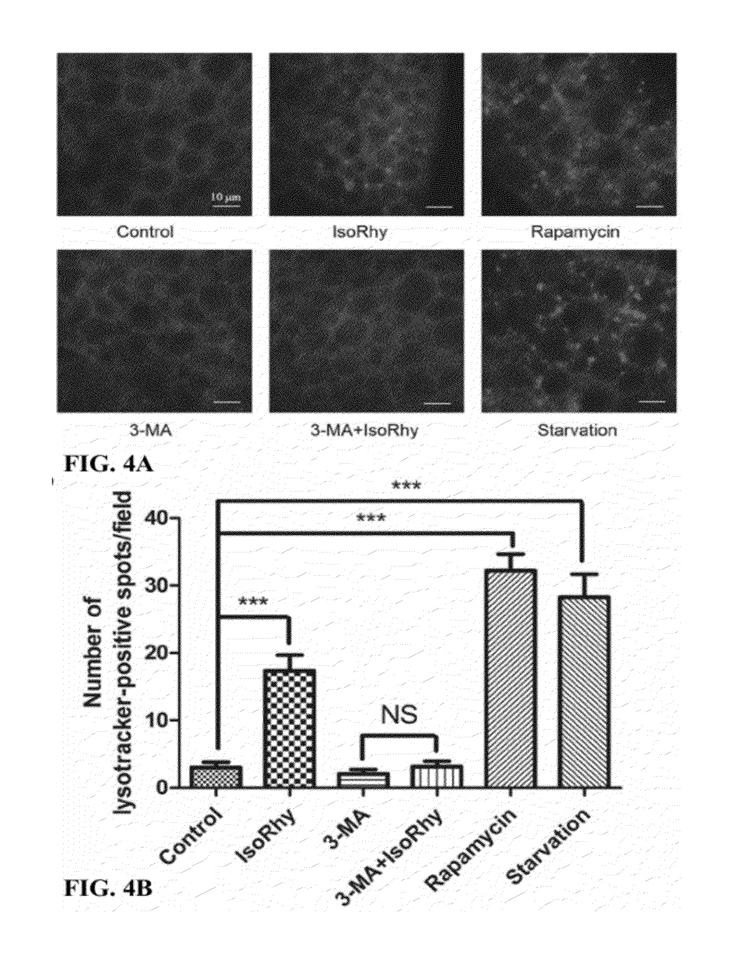

As the major nutrient storage organ of the larvae, the fat body is naturally sensitive to nutrient starvation and elicits a robust autophagic burst upon autophagic stimuli. The basal level of lysosomal activity under nutrient-sufficient conditions is low in fat bodies; however, expansion and acidification of the autolysosome in response to autophagy induction in the fat body can be visualized using the lysotropic dye LysoTracker Red. After 96 hours of egg laying, L3 Drosophila larvae are collected and fed with 0.2 mg/ml of IsoRhy for 6 hours, and fat bodies are then isolated for LysoTracker Red staining. As a positive control, L3 larvae are either fed with 5 μM of rapamycin for 24 hours or starved for 3 hours to induce autophagy. IsoRhy induces formation of LysoTracker Red-positive puncta in the L3 larvae fat bodies, in a similar pattern to rapamycin treatment or starvation (FIG. 4). Furthermore, IsoRhy-induces puncta formation is blocked by autophagy inhibitor 3-MA. These data illustrate that IsoRhy induces autophagy in fat bodies of Drosophila L3 larvae. Accordingly, IsoRhy behaves as a potent autophagy inducer both in vitro and in vivo. Data presents as the mean±SEM of one representative experiment from three independent experiments. (***p<0.001, one-way ANOVA for multiple comparison and Tukey's test as post hoc test).

### Example IV

### IsoRhy Promotes Clearance of Transiently Over-Expressed Pathogenic α-syn Species in N2a Cells Via Autophagy Induction

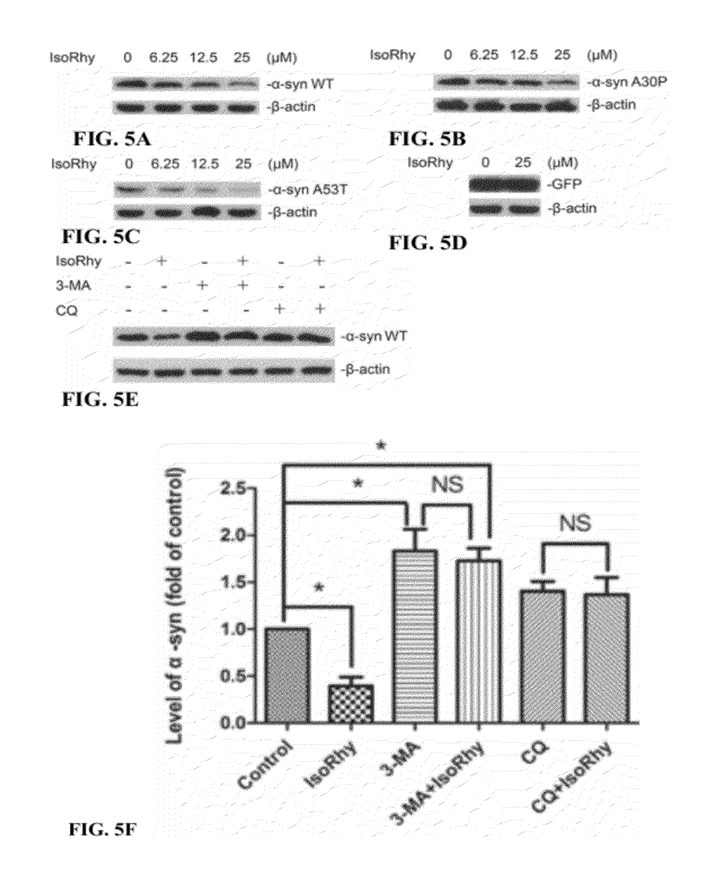

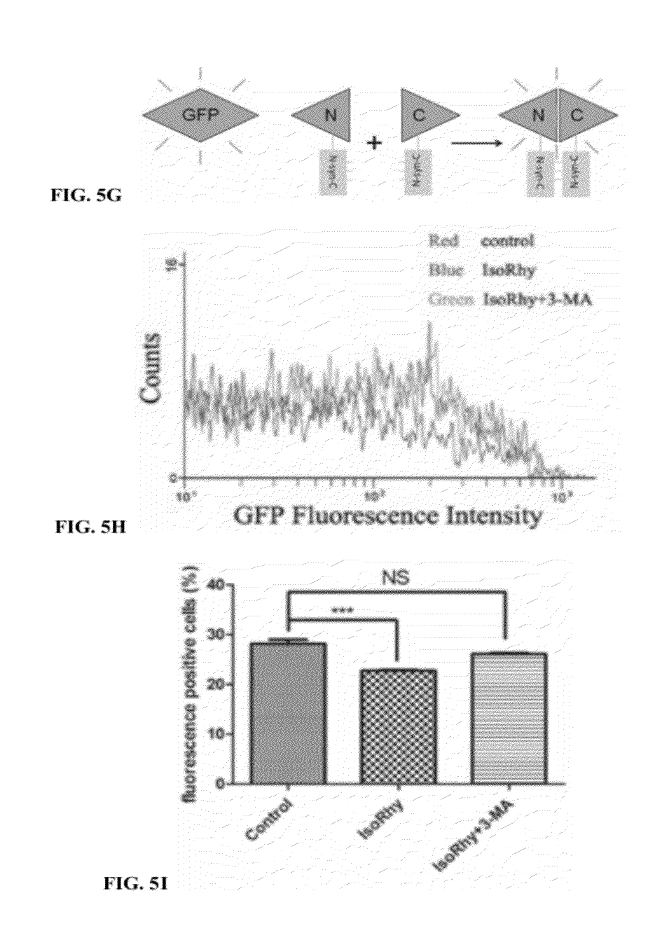

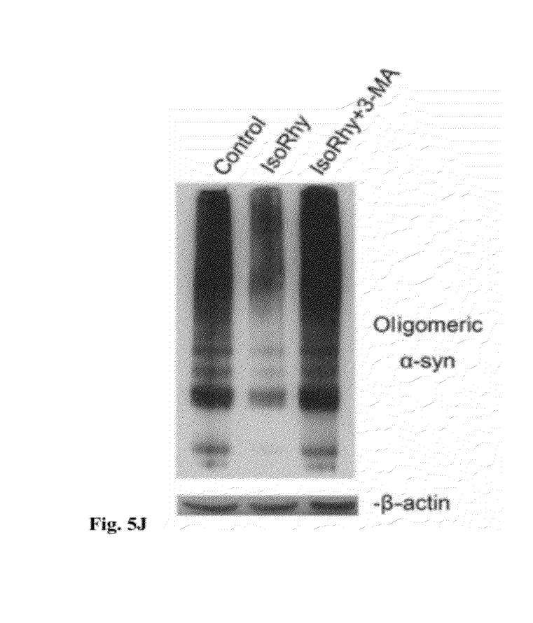

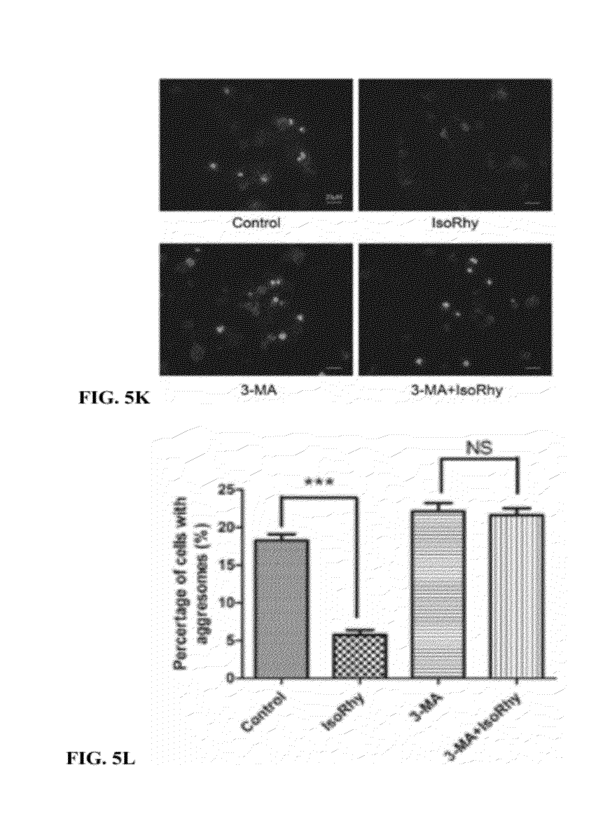

Autophagy has been suggested as a promising therapeutic strategy against synucleinopathies by promoting the clearance of α-syn. IsoRhy induced autophagy-dependent clearance of α-syn is examined in three different cellular models. N2a cells over-expressed in WT and α-syn mutants (A53T and A30P) and N2a cells with GFP as negative control are subjected to IsoRhy treatment for 24 hours. IsoRhy reduces WT and mutant α-syn levels in a dose-dependent manner (FIG. 5A-C), but does not reduce GFP levels (FIG. 5D). Clearance of α-syn induced by IsoRhy is confirmed to be dependent on the autophagy-lysosome pathway because 5 mM autophagy inhibitor 3-MA and 30 μM CQ treatments abolish pro-clearance activity of IsoRhy (FIG. 5E, F). Data presents as the mean±SEM from 3 independent experiments (*p<0.05, one-way ANOVA for multiple comparison and Tukey's test as post hoc test).

Second, the effect of IsoRhy on α-syn oligomers clearance is tested using a bimolecular fluorescence complementation (BiFC)-based cell model which enables visualization of α-syn oligomers. Two non-fluorescent fragments of GFP, GFP-N terminal fraction and GFP-C terminal fraction, are fused with α-syn protein; interaction of the two fragments reconstitutes the fluorophore (FIG. 5G). The presence of fluorescence signal indicates the formation of α-syn oligomers within cells. Mock transfected cells are used as blanks to gate the fluorescence-positive cells and signals stronger than 10¹ RFU are considered as positive α-syn oligomers formation. Cells are treated with 25 μM IsoRhy and/or 5 mM 3-MA for 24 hours and harvested for flow cytometry analysis. IsoRhy promotes degradation of α-syn oligomers as illustrated by decreased fluorescence intensity (FIG. 5H, I) and percentage of cells having high molecular weight α-syn species (FIG. 5J), whereas this effect is prevented by 3-MA Data presents as the mean±SEM from 3 independent experiments (***p<0.001, one-way ANOVA for multiple comparison and Tukey's test as post hoc test).

α-syn and synphilin-1 are co-expressed in N2a cells to mimic aggresome formation and are treated with 25 μM IsoRhy or 5 mM 3-MA for 48 hours. Cells are fixed in 4% paraformaldehyde and analyzed under a fluorescence microscope. IsoRhy dramatically decreases the number of α-syn/synphilin-1 aggresomes as seen in the dramatic decrease of fluorescence intensity observed in the IsoRhy treatment (FIG. 5K, L). The inhibition of IsoRhy clearance of various forms of α-syn and α-syn/synphilin-1 aggresomes in the presence of 3-MA further validates that such clearance is via the autophagy pathway. Data are presented as the mean±SEM from 3 independent experiments (***p<0.001, one-way ANOVA for multiple comparison and Tukey's test as post hoc test).

The results of Example IV shows that autophagy induced by IsoRhy is capable of degrading both the WT and mutated forms of α-syn as well as syn/synphilin-1 aggresomes in neuronal cells, and hence IsoRhy usefulness in treating aggregate-prone disorders.

### Example V

### IsoRhy Promotes the Degradation of α-sym in Human Dopaminergic Neurons Differentiated from Embryonic Stem Cells

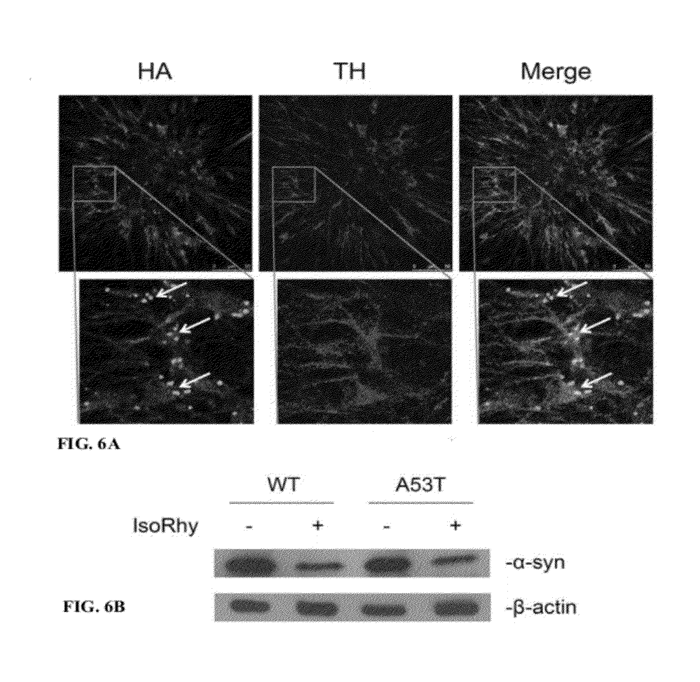

Dopaminergic neurons are the most affected cells in the brains of PD patients, and over-expression of α-syn in the central nervous system leads to dopaminergic neuron degeneration in multiple organisms from mice to C. elegans. IsoRhy promotes α-syn protein degradation via inducing autophagy in N2a cells transiently over-expressing α-syn is shown in FIG. 5. Human embryonic stem cell lines that constitutively express WT and A53T α-syn-HA are established by introducing respective plasmids using lentivirus. The stem cells are then differentiated into DA neurons. The differentiated DA neurons are confirmed by tyrosine hydroxylase (TH) staining, and expression of α-syn is confirmed by HA staining. The massive particles in HA staining images are typical α-syn aggregates (FIG. 6A). The white arrow indicates α-syn aggregates in the cells. As expected, IsoRhy treatment dramatically decreases both WT and A53T α-syn levels in differentiated DA neurons (FIG. 6B). Autophagy promoted by IsoRhy degrades α-syn and mutant thereof in human DA neurons where α-syn is accumulated in PD patients.

### Example VI

### IsoRhy Induces Autophagy in Neuronal Cells in an mTOR-Independent but Beclin-1-Dependent Manner

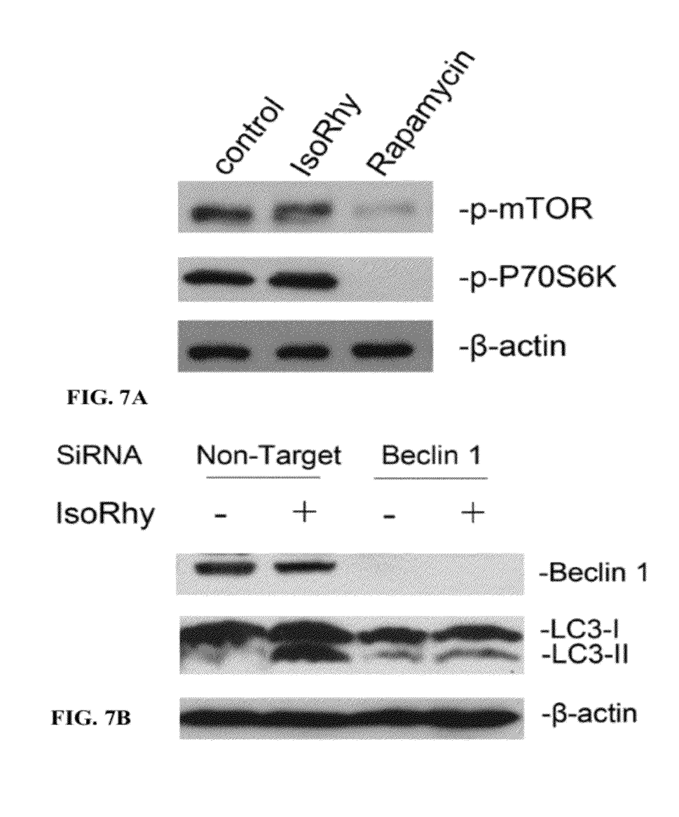

To elucidate the molecular mechanism of IsoRhy action, the classic autophagy controlling pathway, the mTOR pathway is first examined. However, neither phosphorylated mTOR nor its substrate P70S6K are affected by IsoRhy treatment, although phosphorylated mTOR and pP70S6K were dramatically inhibited by rapamycin (FIG. 7A). Several other pathways reported to be involved in the activation of autophagy, including AKT, AMPK, MEK/ERK, JNK, and ER stress pathways, and calcium signaling pathway (data not shown), are also tested. However, none of the above mentioned pathways are altered by IsoRhy treatment. Beclin-1 is a key player in the activation of autophagy, and up-regulated Beclin-1 has been shown to directly induce autophagy. To understand the role of Beclin-1 in IsoRhy-induced autophagy in neuronal cells, the effect of IsoRhy in the presence or absence of Beclin-1 depletion by RNA interference is examined. Data reveals that IsoRhy does not affect the expression of Beclin-1, but Beclin-1 siRNA treatment completely blocks IsoRhy-induced autophagy (FIG. 7B). These data indicate that IsoRhy induces autophagy in neuronal cells in an mTOR-independent but Beclin-1-dependent manner.

### Example VII

### Tetracyclic Oxindole Alkaloids Induce Autophagy in Neuronal Cells

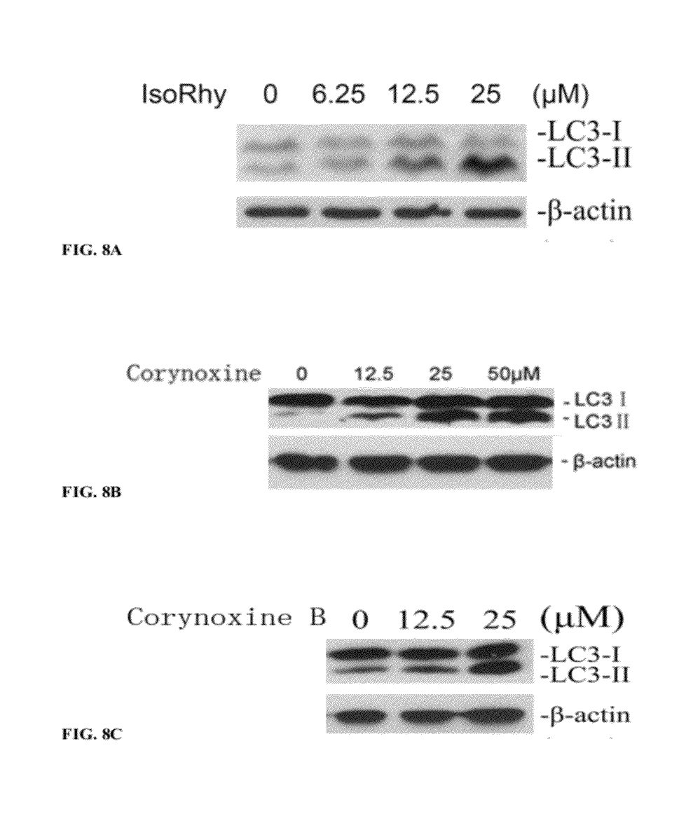

To compare the activities of oxindole alkaloids on autophagy induction, LC3II expression levels in N2a cells after treatment with Isorhynchophylline (IsoRhy) (FIG. 8A), Corynoxine (Cory) (FIG. 8B) and Corynoxine B (Cory B) (FIG. 8C) for 12 hours are examined. The data reveals that Isorhy, Cory and Cory B significantly activate autophagy in N2a cells, demonstrating that tetracyclic oxinodole alkaloids are excellent autophagy inducers.

### Results

Based on the working examples, it is demonstrated that pro-autophagy activity of IsoRhy is highly responsive in neuronal cells. It induces substantial autophagy in a wide range of neuronal cell lines (N2a, SH-SY5Y and PC12) as well as in primary neuron cultures as illustrated by the increase of LC3-II/actin ratio and GFP-LC3 puncta formation. While it is well-known that α-syn can be degraded either by proteasomes, macroautophay and chaperone-mediated autophagy (CMA), only the two autophagy pathways are capable of degrading α-syn. More specifically, it has been reported that mutant α-syn inhibits CMA and only macroautophagy can degrade mutant α-syn. The working examples show that IsoRhy specifically enhances macroautophagy and significantly degrades WT, mutant alpha-synuclein monomers, alpha-synuclein oligomers as well as alpha-synuclein/synphilin-1 aggresomes in different human DA cells which is not shown in previous chemical autophagy inducers like rapamycin, trehalose and 17-AAG. The mTOR independent autophagy-inducing effect of IsoRhy demonstrated also means that treatment of diseases that benefit from autophagy with the present invention eliminates any side-effects or complications related to the mTOR pathway.

Lastly, Drosophila larvae have proven to be useful in exploring the molecular mechanisms as well as the physiological functions of autophagy in vivo. Therefore, the demonstration of the present invention that IsoRhy is capable of inducing autophagy in drosophila L3 larvae fat body illustrates in vivo autophagy induction ability thereof.

### INDUSTRIAL APPLICABILITY

The present invention discloses novel compositions including IsoRhy and tetracyclic oxindole alkaloids that induce autophagy in neurons to degrade protein aggregates independent of mTOR both in vivo and in vitro and the application thereof in treating diseases that can benefit from autophagy inducement and free from mTOR associated complications.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

What we claim:

1. A composition for inducing autophagy for the treatment of disease that can benefit from autophagy inducement comprising a therapeutically effective amount of a compound of formula (I-V) or a pharmaceutically acceptable salt thereof: 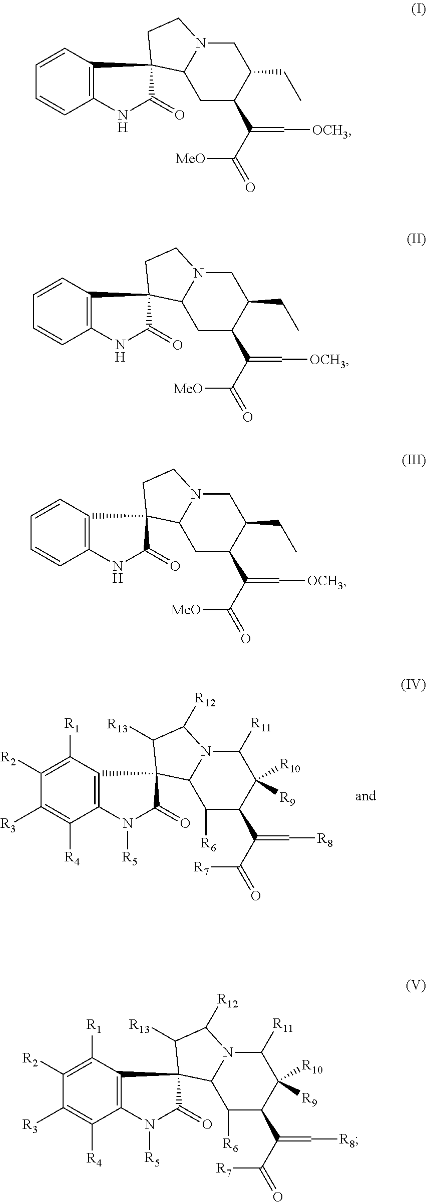
wherein R₁, R₂, R₃, R₄, R₅, R₆, R₁₁, R₁₂ and R₁₃ are each independently selected from hydrogen, hydroxyl, halogen, C1-6 alkyl and C1-6 haloalkyl;
R₇ and R₈ are each independently selected from methoxyl and hydroxyl;
R₉ and R₁₀ are each independently selected from hydrogen, hydroxyl, halogen, C1-6 alkyl and wherein the compounds are small enough to pass through the blood brain barrier of a mammalian brain.

2. The composition according to claim 1, wherein said compounds are tetracyclic oxindole alkaloids isolated from Uncariae species or chemically synthesized.

3. The composition according to claim 1, wherein said disease comprises Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, neuronal intranuclear hyaline inclusion disease and cancer, and other related symptoms and/or conditions thereof.

4. The composition of claim 1, wherein said composition degrades abnormal protein aggregates in or among neurons and said abnormal protein aggregates comprises aggregates of alpha-synuclein, huntinting, tau, SOD1, PMP22 or variants and mutated forms thereof.

5. The composition of claim 2, wherein said tetracyclic oxindole alkaloids comprises isorhynchophylline (formula I), corynoxine (formula II) and corynoxine B (formula III).

6. The composition according to claim 4, wherein said alpha-synuclein comprises wild-type and mutant alpha-synuclein monomers, wild-type and mutant alpha-synuclein oligomers, and wild-type and mutant alpha-synuclein/synphilin-1 aggresomes.

7. The composition according to claim 1, wherein said composition is administered in conjunction with one or more other therapeutic agent that treat diseases that can benefit from autophagy inducement or potentiate the autophagy inducing activity of compounds of formula (I-V).

8. The composition of claim 1, wherein said composition further comprises one or more of a pharmaceutically acceptable carrier, solvent, excipient, adjuvant, prodrug, and other therapeutic agent that treat diseases that can benefit from autophagy inducement or potentiate the autophagy inducing activity of said compounds.

9. The composition of claim 1, wherein said composition is administered to a subject in need thereof via oral administration, intravenous injection, intravenous infusion, intra-peritoneal injection, intramuscular injection and/or subcutaneous injection.

10. The composition according to claim 1, wherein said composition is in a form comprising solution, solid, tablet, capsule, powder, paste and aerosol.

11. A method for inducing autophagy for the treatment of disease that can benefit from autophagy inducement comprising administering a pharmaceutical composition to a subject in need thereof, said pharmaceutical composition comprising a therapeutically effective amount of a compound of formula (I-V) or a pharmaceutically acceptable salt thereof: 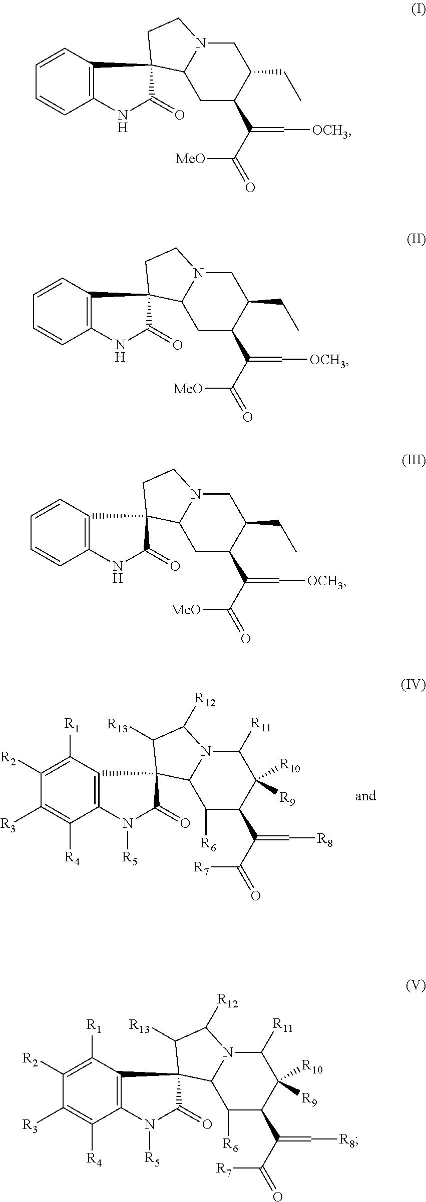
wherein R₁, R₂, R₃, R₄, R₅, R₆, R₁₁, R₁₂ and R₁₃ are each independently selected from hydrogen, hydroxyl, halogen, C1-6 alkyl and C1-6 haloalkyl;
R₇ and R₈ are each independently selected from methoxyl and hydroxyl;
R₉ and R₁₀ are each independently selected from hydrogen, hydroxyl, halogen, C1-6 alkyl and wherein the compound is small enough to pass through the blood brain barrier of a mammalian brain.

12. The method according to claim 11, wherein said composition further comprises one or more of a pharmaceutically acceptable carrier, solvent, excipient, adjuvant, prodrug, and other therapeutic agent that treat diseases that can benefit from autophagy inducement or potentiate the autophagy inducing activity of said compound.

13. The method according to claim 11, wherein said method further comprising administering one or more other therapeutic agent that treat diseases that can benefit from autophagy inducement or potentiate the autophagy inducing activity of compound of formula (I-V) to the subject in need thereof.

14. The method according to claim 11, wherein said administering comprises administering said composition to the subject in need thereof via oral administration, intravenous injection, intravenous infusion, intra-peritoneal injection, intramuscular injection and/or subcutaneous injection.

15. The method according to claim 11, wherein said compounds are tetracyclic oxindole alkaloids isolated from Uncariae species or chemically synthesized.

16. The method according to claim 11, wherein said disease comprises Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, neuronal intranuclear hyaline inclusion disease and cancer, and other related symptoms and/or conditions thereof.

17. The method according to claim 15, wherein said tetracyclic oxindole alkaloids comprises isorhynchophylline (formula I), corynoxine (formula II) and corynoxine B (formula III).

18. The method according to claim 11, wherein said composition is in a form comprising solution, solid, tablet, capsule, powder, paste and aerosol

19. A method of preparing a pharmaceutical composition for autophagy inducement in treating a disease that can benefit from autophagy inducement comprising forming the composition from a compound of formula (I-V) or a pharmaceutically acceptable salt thereof in preparation of a pharmaceutical composition for autophagy induction in treating diseases that can benefit from autophagy inducement: 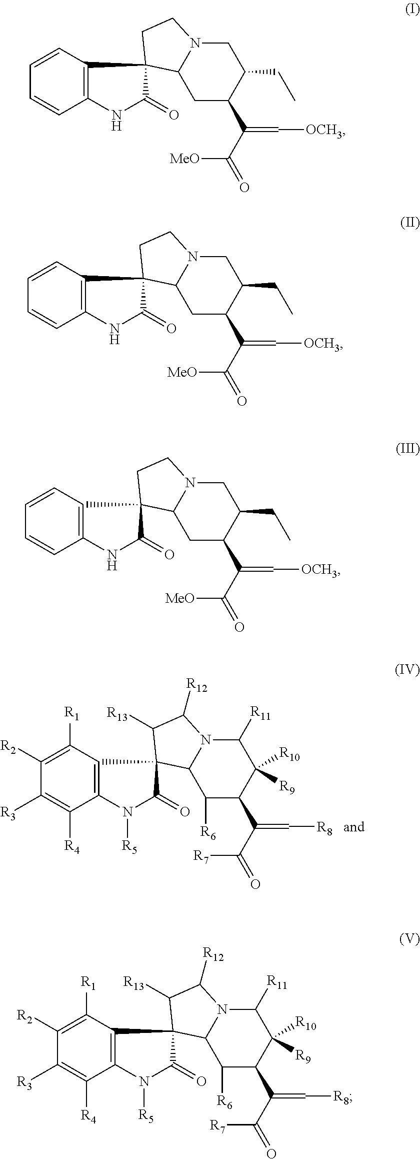
wherein R₁, R₂, R₃, R₄, R₅, R₆, R₁₁, R₁₂ and R₁₃ are each independently selected from hydrogen, hydroxyl, halogen, C1-6 alkyl and C1-6 haloalkyl;
R₇ and R₈ are each independently selected from methoxyl and hydroxyl;
R₉ and R₁₀ are each independently selected from hydrogen, hydroxyl, halogen, C1-6 alkyl and which are mTOR-independent and beclin1-dependent autophagy inducers to degrade abnormal protein aggregates in or among neurons and is capable of promoting autophagosome maturation process.

20. The method of claim 19, wherein said compounds are tetracyclic oxindole alkaloids isolated from Uncariae species or chemically synthesized.
